(19) United States
(12) Patent Application Publication
(10) Pub. No.: US 2024/0335573 A1
(43) Pub. Date: Oct. 10, 2024

(54) RADIONUCLIDE MICROSPHERES AND PREPARATION METHOD THEREFOR AND APPLICATION THEREOF

(71) Applicant: Suzhou Haowei Medical Technology Co., Ltd., Suzhou (CN)

(72) Inventors: Ping GUO, Suzhou (CN); Pingping CHEN, Suzhou (CN)

(73) Assignee: Suzhou Haowei Medical Technology Co., Ltd., Suzhou, JS (CN)

(21) Appl. No.: 18/627,406

(22) Filed: Apr. 4, 2024

(30) Foreign Application Priority Data
Apr. 4, 2023 (CN) 202310350883.8

(51) Int. Cl. A61K 51/12; A61K 51/06; A61K 51/10

(52) U.S. Cl. CPC A61K 51/1255; A61K 51/065; A61K 51/103; A61K 51/1096

(57) ABSTRACT

Disclosed are radionuclide microspheres and a preparation method therefor and an application thereof. The radionuclide microspheres include at least one or more radionuclides and are microspheres loading the radionuclides. The preparation method includes: by using an improved emulsion polymerization process, forming a prepolymer intermediate by a structural monomer, a functional monomer, and a vinyl crosslinker; adding one or more radionuclides for coordination polymerization with the prepolymer intermediate to form nuclear particles; and adding one or more small molecule monomers or high molecular materials for secondary polymerization to obtain the radioactive microspheres. The radionuclide microspheres are negatively charged porous microspheres which can load both chemotherapeutic drugs and polypeptide biological drugs. The microspheres may be implanted via vascular intervention and percutaneous puncture and may be used for treating solid malignant tumors such as liver cancer and lung cancer.

### BACKGROUND

### Technical Field

The present disclosure relates to radionuclide microspheres, further to a preparation method of the radionuclide microspheres and an application thereof, and belongs to the technical field of medical materials.

### Related Art

Malignant tumors, for example, liver cancer, are the first killer threatening human health. They are not only high in incidence rate, but also pernicious and refractory, and cause a high death rate. A radiotherapy, one of important methods to treat malignant tumors, inhibits and destroys tumor cells by means of rays and ionizing radiation emitted by radionuclides to achieve a goal of treatment. The selective internal radiation therapy (SIRT) means radiotherapy by injecting drugs containing the radionuclides into a human body or pressing instruments close to target tissues or inserting the instruments into the target tissues. Radioactive substances are selectively delivered to tumor tissues with a large radiation dosage to the tumor tissues. The amount of radioactive substances entering surrounding tissues is very small, so that the damage to normal tissues is very small. The SIRT may be divided into a general radionuclide therapy, a radionuclide targeted therapy, and a radionuclide interventional therapy according to different methods. The radionuclide interventional therapy may further include a radioactive scaffold, a radioactive seed source, radioactive microspheres, and other forms which may be directly implanted into the tumor tissues according to different carriers. The radioactive microspheres refer to microspheres prepared from radionuclides which release B or low energy y rays and are suitable for therapy and matrixes such as glass and resins, where the diameter of the microspheres is 5-200 μm. At present, glass microspheres and resin microspheres are still the main path to prepare the radioactive microspheres. A preparation process of the glass microspheres includes: mixing ⁸⁹Y with super purity aluminum oxide and silicon dioxide to obtain a mixture; smelting the mixture in a 1500° C. smelting furnace; after cooling the mixture, embedding ⁸⁹Y into glass and crushing the mixture, and smelting and “nodulizing” glass particles by a flamethrower; and then bombarding the spheres with neutrons to transform embedded ⁸⁹Y into ⁹⁰Y. This method is complicated in process and high in production cost. Moreover, the glass microspheres have a high density as a glass matrix is used. A preparation process of the resin microspheres includes: loading ⁹⁰Y nuclide onto the resin microspheres, and settling and curing the resin microspheres to obtain the resin microspheres. However, the radionuclide microspheres prepared by this method has a relatively low specific activity, so that their promotion and application are limited to a certain extent.

Hydrogel microspheres, as a novel high molecular polymer material, have developed rapidly in recent years, and quite a few ground-breaking research results have been achieved. This is because that the hydrogel microspheres may be applied to target delivery and controlled release of drugs, gene transfection, medical diagnosis, biosensors, isolation and purification of biological substances and the like. In view of a plenty of clearance space and pores in a hydrogel microsphere system, for example, the clearance space among accumulated hydrogel microspheres and pores in the hydrogel microsphere particles, the hydrogel microspheres have relatively strong adsorption to material particles (for example, metal ions and drug molecules). Therefore, the hydrogel microspheres may be used as an excellent sustained-release drug delivery system.

To sum up, the glass microspheres feature a high density, a high requirement on purity of raw materials, a complicated preparation process, dependence on a reactor and the like, so that the radioactive glass microspheres are limited in application. The density of the resin microspheres is still higher than blood density and the specific activity of the radioactive resin microspheres is not high, so that the clinical application is limited to a certain extent. Therefore, it is necessary to improve the material and structure of a radionuclide therapy carrier to establish a simpler preparation process, so as to reduce the density of the radionuclide microspheres, improve the specific activity of the radionuclide microspheres, and meet the requirement on using performance of the carrier in the radionuclide interventional therapy.

### SUMMARY

To solve the problems in the related art, the present disclosure chiefly provides radionuclide microspheres.

The present disclosure further provides a preparation method of the radionuclide microspheres and an application thereof.

To achieve the object, the present disclosure provides the following technical solution:

According to a first aspect of embodiments of the present disclosure, provided are radionuclide microspheres, including at least one or more radionuclides, which are microspheres loading the radionuclides. The radionuclide microspheres are formed by polymerizing nuclear particles and one or more small molecule monomers or high molecular materials, the nuclear particles being formed by coordination polymerization of radionuclides and a prepolymer intermediate; and the prepolymer intermediate is formed by polymerizing a structural monomer, a functional monomer and a vinyl crosslinker, where the mass ratio of the structural monomer, the functional monomer, and the vinyl crosslinker is 1:(0.01-8):(0.01-2); and the particle size of the microspheres is 5-200 μm.

According to the radionuclide microspheres described above, preferably, the structural monomer is a compound containing hydrophilic functional groups such as hydroxyl, amino and carboxyl, more preferably one or more of acrylamide, methacrylamide, acrylic acid, methacrylic acid, hydroxyethyl acrylate, hydroxyl propyl acrylate, halogenated acrylic acid, and halogenated methacrylic acid.

According to the radionuclide microspheres described above, preferably, the functional monomer is an organic salt with carboxylic acid groups or sulfonic acid groups, more preferably one or more of sodium acrylate, acryloylamino sodium ethyl carboxylate, 2-acrylamide-2-methyl sodium propyl carboxylate, and 2-acrylamide-2-methyl sodium propanesulfonate.

According to the radionuclide microspheres described above, preferably, the vinyl crosslinker is a water soluble compound, more preferably one or more of N,N′-methylene diacrylamide, N,N′-ethylenebisacrylamide, polyethylene glycol diacrylate, polypropylene glycol acrylate, 3-arm-polyethylene glycol-acrylamide, 3-arm-polyethylene glycol-acrylate, 4-arm-polyethylene glycol-acrylamide, and 4-arm-polyethylene glycol-acrylate.

According to the radionuclide microspheres described above, preferably, the small molecule monomer or high molecular material is a water soluble compound. The small molecule monomer is preferably one or more of acrylamide, phosphonitrile, and dopamine; and the high molecular material is formed by a reaction on one or more of gelatin, sodium alginate, sodium hyaluronate, carboxymethyl chitosan, polyvinyl alcohol, polythreonine and polyserine, and butanediol diglycidyl ether, polyethylene glycol diglycidyl ether or polypropylenglycol diglycidyl ether.

According to the radionuclide microspheres described above, preferably, the radionuclide is selected from at least one of lanthanum, yttrium, technetium, strontium, praseodymium, samarium, europium, gadolinium, terbium, zirconium, holmium, erbium, ytterbium, lutecium, rhenium, and gallium.

According to a second aspect of the embodiments of the present disclosure, provided is a preparation method of radionuclide microspheres, including the following steps:
performing a reaction on the structural monomer, the functional monomer, and the vinyl crosslinker to prepare the prepolymer intermediate;
performing coordination polymerization on metal ions of the radionuclides in the prepolymer intermediate through a coordination interaction between the prepolymer intermediate and the one or more radionuclides to prepare nuclear particles; and
performing a secondary polymerization reaction on the nuclear particles and the one or more small molecule monomers or high molecular materials to obtain a product, and cleaning, filling, and sterilizing the product to prepare the radionuclide microspheres.

According to the preparation method described above, preferably, the steps are specifically operated as follows:
S1: placing an emulsifier in an oil phase solvent to form an oil phase solution, the oil phase solvent including: petroleum ether, n-heptane, cyclohexane and/or liquid paraffin;
S2: mixing the structural monomer, the functional monomer, the vinyl crosslinker and an initiator with water to form a mixed aqueous solution;
S3: dropwise adding the mixed aqueous solution prepared in S2 into the oil phase solution prepared in S1 in a stirring condition to react to prepare the prepolymer intermediate;
S4: dropwise adding the solution containing the radionuclide ions into the prepolymer intermediate prepared in S3 in a stirring condition, and dropwise adding a catalyst tetramethylethylenediamine to react to prepare the nuclear particles;
S5: dropwise adding the small molecule monomers or high molecular materials into the nuclear particle solution prepared in S4 in a stirring condition for a further secondary polymerization reaction to obtain a product, and cleaning, filling, and sterilizing the product to prepare the radionuclide microspheres.

According to the preparation method described above, preferably, the emulsifier is selected from one or more of OP-10, EM90, span-60, and tween-80.

According to the preparation method described above, preferably, the mass ratio of the emulsifier to the oil phase solvent is 1:(20-1000).

According to the preparation method described above, preferably, the initiator includes one or more of potassium persulfate, sodium persulfate, and ammonium persulfate.

According to the preparation method described above, preferably, the structural monomer includes one or more of acrylamide, methacrylamide, acrylic acid, methacrylic acid, hydroxyethyl acrylate, hydroxyl propyl acrylate, halogenated acrylic acid, and halogenated methacrylic acid.

According to the preparation method described above, preferably, the functional monomer includes one or more of sodium acrylate, acryloylamino sodium ethyl carboxylate, 2-acrylamide-2-methyl sodium propyl carboxylate, and 2-acrylamide-2-methyl sodium propanesulfonate.

According to the preparation method described above, preferably, the vinyl crosslinker includes one or more of N,N′-methylene diacrylamide, N,N′-ethylenebisacrylamide, polyethylene glycol diacrylate, polypropylene glycol acrylate, 3-arm-polyethylene glycol-acrylamide, 3-arm-polyethylene glycol-acrylate, 4-arm-polyethylene glycol-acrylamide, and 4-arm-polyethylene glycol-acrylate.

According to the preparation method described above, preferably, the mass ratio of the structural monomer, the functional monomer, the vinyl crosslinker, and the initiator is 1:(0.01-8):(0.01-2):(0.01-1).

According to the preparation method described above, preferably, in S2, the mass ratio of the total mass of the structural monomer, the functional monomer, the vinyl crosslinker, and the initiator to water is 1:(0.3-3).

According to the preparation method described above, preferably, the mass ratio of the mixed solution prepared in S2 to the oil phase solution prepared in S1 is 1:(8-15).

According to the preparation method described above, preferably, the metal ions of the radionuclide in S4 include, but are not limited to, lanthanum (La³⁺), yttrium (Y³⁺), technetium (Tc⁴⁺), strontium (Sr²⁺), praseodymium (Pr³⁺), samarium (Sm³⁺), europium (Eu³⁺), gadolinium (Gd³⁺), terbium (Tb³⁺), zirconium (Zr⁴⁺), holmium (Ho³⁺), erbium (Er³⁺), ytterbium (Yb³⁺), lutetium (Lu³⁺), rhenium (Re³⁺), and gallium (Ga²⁺), and other elements similar to the above metal elements in chemical behavior and all various nonradioactive and radioactive isotopes thereof.

According to the preparation method described above, preferably, the small molecule monomer in S5 is one or more of acrylamide, phosphonitrile, and dopamine. The mass ratio of the nuclear particle solution prepared in S4 to the small molecule monomers is 1:(0.1-20). The high molecular material is formed by the reaction on one or more of gelatin, sodium alginate, sodium hyaluronate, carboxymethyl chitosan, polyvinyl alcohol, polythreonine and polyserine, and butanediol diglycidyl ether, polyethylene glycol diglycidyl ether or polypropylenglycol diglycidyl ether; and the mass ratio of the nuclear particle solution prepared in S4 to the high molecular materials is 1:(1-20).

In another aspect, the present disclosure provides radionuclide microspheres, prepared by the method described above.

According to the radionuclide microspheres prepared by the method provided by the present disclosure, preferably, the diameter of the radionuclide microspheres is 5-200 μm, preferably 20-80 μm, and more preferably 20-50 μm.

The particle size of the radionuclide microspheres prepared by the present disclosure may be adjusted in the range, and in most medical applications, microspheres with narrow particle size distribution are needed to facilitate selective radiation therapy. The radionuclide microspheres prepared by the present disclosure have the advantages of adjustable particle size and narrow particle size distribution, so that the problem that the size range of the microspheres is controlled by different screening methods in the related art is solved.

According to a third aspect of the embodiments of the present disclosure, provided is an application of radionuclide microspheres in preparing drugs for treating tumors. The radionuclide microspheres are the above radionuclide microspheres or the radionuclide microspheres prepared by the above preparation method.

Compared with the related art, the present disclosure has the beneficial effects:
(1) A matrix of the radionuclide microspheres prepared by the present disclosure includes acrylic acid, acrylamide or polyvinyl alcohol high molecular materials and features super water absorption, biocompatibility, and mechanical strength. The radionuclide microspheres feature a good suspension property in blood when the density is appropriate, which facilitates injection and delivery of the microspheres to a target position accurately.
(2) The nuclear particles in the present disclosure are formed by the coordination polymerization reaction on the radionuclides and the functional monomer (the organic salt containing carboxylic acid groups or sulfonic acid groups) through the coordination interaction. During coordination polymerization, to regulate and control the mass ratio of the functional monomer may adjust the loading capacity of the radionuclides at the centers of the nuclear particles. Moreover, homonuclear or heteronuclear radionuclides are added in the coordination polymerization reaction to form homonuclear or heteronuclear nuclear particles, so that a purpose that the microspheres load one or more radionuclides may be achieved. The loading capacity of the radionuclides by the microspheres is substantially 0.001-5 Ci/g microspheres.
(3) In the present disclosure, hydrophilic small molecular or high molecular materials are used to perform the secondary crosslinking polymerization reaction on the radioactive nuclear particles, so that the radioactive nuclear radionuclides are in a stable state in the microspheres. The secondary crosslinking reaction centered by the nuclear particles provides abundant network structures to the interior of the microspheres, so that dropping or leaching of radionuclide ions in the microspheres may be further inhibited. The lowest 30-day drop rate of the gel microspheres may be less than 0.01%.
(4) The radionuclide microspheres prepared by the present disclosure may further load chemotherapeutic drugs such as doxorubicin, pharmorubicin, pirarubicin, irinotecan, bleomycin, and gemcitabine or polypeptide biological drugs such as bevacizumab and PD-1. The drug loading ratio of the radionuclide microspheres loading the chemotherapeutic drugs or the biological drugs may reach 95-99%.
(5) The particle size of the radionuclide microspheres prepared by the present disclosure is adjustable within a range of 5-200 μm and particle size distribution is narrow, so that the limitation that the size range of the microspheres is controlled by different screening methods in the related art is overcome.
(6) The radionuclide microspheres prepared by the present disclosure feature a simple preparation process, mild reaction conditions, controllable time, high safety, a low cost, and the like.
(7) The microspheres may be implanted via vascular intervention and percutaneous puncture and may be used for treating solid malignant tumors such as liver cancer and lung cancer, with a good curative effect.

### BRIEF DESCRIPTION OF THE DRAWINGS

FIG. 1 is an optical microphotograph of radionuclide microspheres in an example 1;

FIG. 2 is an optical microphotograph of radionuclide microspheres in an example 2;

FIG. 3 is an optical microphotograph of radionuclide microspheres in an example 3;

FIG. 4 is an optical microphotograph of radionuclide microspheres in an example 7;

FIG. 5 is an optical microphotograph of radionuclide microspheres in an example 8; and

FIG. 6 is an optical microphotograph of radionuclide microspheres in an example 9.

### DETAILED DESCRIPTION

The technical content of the present disclosure will be described in detail below in combination with drawings and specific examples.

### Example 1 Preparation of Y-90 Loading Microspheres

(1) An emulsifier span-60 was added into petroleum ether to form an oil phase solution, where the mass percentage of the emulsifier in the oil phase solution was 0.1 wt %.

Acrylamide, 2-acrylamide-2-methyl sodium proparesulfonate, polyethylene glycol diacrylate, potassium persulfate, and tetramethyl ethylenediamine were weighed at the mass ratio of 30:60:5:2:3.

Acrylamide, 2-acrylamide-2-methyl sodium proparesulfonate, polyethylene glycol diacrylate, and potassium persulfate were mixed with water to form a mixed aqueous solution, where the mass percentage of water in the mixed aqueous solution was 65%.

The mixed aqueous solution was dropwise added into an oil phase solution in a stirring condition to obtain a mixture, the mass ratio of the mixed aqueous solution to the oil phase solution being 1:10, and the mixture was reacted at 40° C. for 2 h to form a prepolymer intermediate.
(2) A 1 mCi ⁹⁰YCl₃ solution was added into the prepolymer intermediate dispersion obtained in S(1), and tetramethyl ethylenediamine was dropwise added at 50° C. to react for 3 h to obtain ⁹⁰Y nuclear particles.
(3) The nuclear particles in S(2), sodium alginate, polyethylene glycol bisglycidyl ether, and a sodium hydroxide solution (concentration was 5 wt %) were weighed at the mass ratio of 1:20:2:5.

The nuclear particles in S(2) were added into a solution where sodium alginate was uniformly mixed with sodium hydroxide in advance, polyethylene glycol diglycidyl ether was then added into the mixed solution, the mixed solution was heated to 40° C., after the mixed solution was stirred to react for 2 h, the mixed solution was kept standing for 30 min, and the mixed solution was filtered and washed; and the mixed solution was filled and sterilized to prepare 20-80 μm radionuclide ⁹⁰Y microspheres (the yield of the microspheres was 40%, the radioactive activity value was 0.98 mCi, the labeling rate of nuclide ⁹⁰Y was 98%, the density was 1.02 g/cm³, the BET specific surface area of the microspheres was 6.5 m²/g, and the BJH average pore diameter of the microspheres was 24.7 nm).

Infrared IR analysis (KBr pellet, cm⁻¹): 3436, 2931, 1614, 1418, 1303, 1107, 1036, 620.

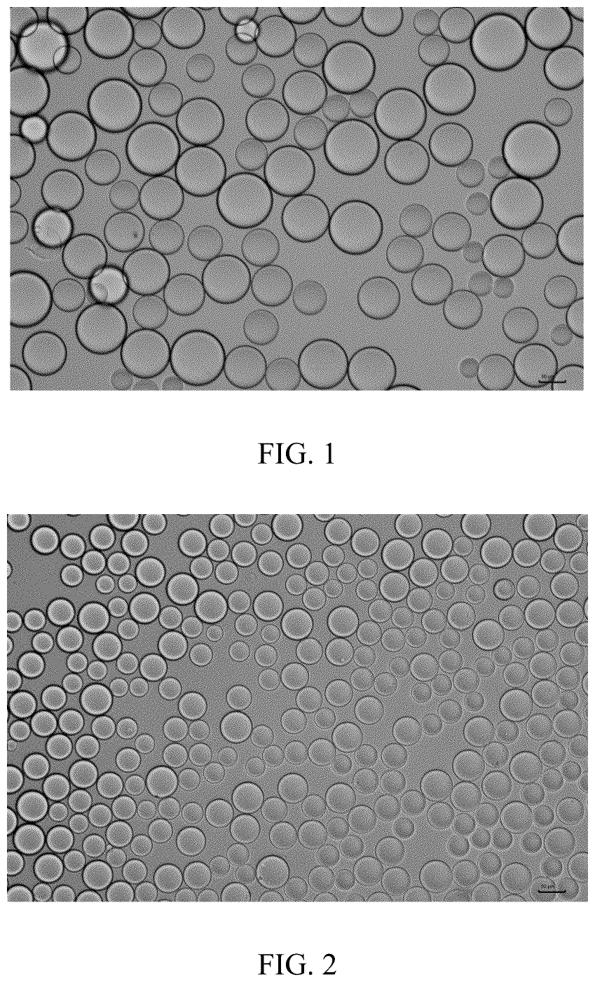

FIG. 1 is an optical microphotograph of radionuclide ⁹⁰Y microspheres obtained. It could be seen from the microphotograph that the particle size of the microspheres is relatively uniform, and the microspheres are spheroidal.

### Example 2 Preparation of Y-90 Loading Microspheres

(1) An emulsifier tween-80 was added into petroleum ether to form an oil phase solution, where the mass percentage of the emulsifier in the oil phase solution was 0.2 wt %.

Hydroxyethyl acrylate, sodium acrylate, N,N′-methylene diacrylamide, ammonium persulfate, and tetramethyl ethylenediamine were weighed at the mass ratio of 35:55:4:3:3.

Hydroxyethyl acrylate, sodium acrylate, N,N′-methylene diacrylamide, and ammonium persulfate were mixed with water to form a mixed aqueous solution, where the mass percentage of water in the mixed aqueous solution was 50%.

The mixed aqueous solution was dropwise added into an oil phase solution in a stirring condition to obtain a mixture, the mass ratio of the mixed aqueous solution to the oil phase solution being 1:12, and the mixture was reacted at 30° C. for 2 h to form a prepolymer intermediate.
(2) A 5 Ci ⁹⁰YCl₃ solution was added into the prepolymer intermediate dispersion obtained in S(1), and tetramethyl ethylenediamine was dropwise added at 40° C. to react for 4 h to obtain ⁹⁰Y nuclear particles.
(3) The nuclear particles in S(2), carboxymethyl chitosan, butanediol diglycidyl ether, and a sodium hydroxide solution (concentration was 5 wt %) were weighed at the mass ratio of 1:20:2:5.

The nuclear particles in S(2) were added into a solution where carboxymethyl chitosan was uniformly mixed with sodium hydroxide in advance, butanediol diglycidyl ether was then added into the mixed solution, the mixed solution was heated to 30° C., after the mixed solution was stirred to react for 3 h, the mixed solution was kept standing for 30 min, and the mixed solution was filtered and washed; and the mixed solution was filled and sterilized to prepare 20-50 μm radionuclide ⁹⁰Y microspheres (the yield of the microspheres was 58%, the radioactive activity value was 4.96 Ci, the labeling rate of nuclide ⁹⁰Y was 99%, the density was 1.03 g/cm³, the BET specific surface area of the microspheres was 9.6 m2/g, and the BJH average pore diameter of the microspheres was 18.3 nm).

Infrared IR analysis (KBr pellet, cm⁻¹): 3413, 2916, 1591, 1425, 1310, 1092, 705.

FIG. 2 is an optical microphotograph of radionuclide ⁹⁰Y microspheres obtained. It could be seen from the microphotograph that the particle size of the microspheres is relatively uniform, and the microspheres are spheroidal.

### Example 3 Preparation of Lu-177 Loading Microspheres

(1) An emulsifier EM 90 was added into petroleum ether to form an oil phase solution, where the mass percentage of the emulsifier in the oil phase solution was 0.1 wt %.

Hydroxyethyl acrylate, 2-acrylamide-2-methyl sodium proparesulfonate, 3-arm-polyethylene glycol-acrylate, ammonium persulfate, and tetramethyl ethylenediamine were weighed at the mass ratio of 30:60:8:1:1.

Hydroxyethyl acrylate, 2-acrylamide-2-methyl sodium proparesulfonate, 3-arm-polyethylene glycol-acrylate, and ammonium persulfate were mixed with water to form a mixed aqueous solution, where the mass percentage of water in the mixed aqueous solution was 50%.

The mixed aqueous solution was dropwise added into an oil phase solution in a stirring condition to obtain a mixture, the mass ratio of the mixed aqueous solution to the oil phase solution being 1:8, and the mixture was reacted at 30° C. for 2 h to form a prepolymer intermediate.
(2) A 2 mCi ¹⁷⁷LuCl₃ solution was added into the prepolymer intermediate dispersion obtained in S(1), and tetramethyl ethylenediamine was dropwise added at 40° C. to react for 4 h to obtain ¹⁷⁷Lu nuclear particles.
(3) The nuclear particles in S(2), carboxymethyl chitosan, polypropylenglycol diglycidyl ether, and a sodium hydroxide solution (concentration was 5 wt %) were weighed at the mass ratio of 1:20:2:5.

The nuclear particles in S(2) were added into a solution where carboxymethyl chitosan was uniformly mixed with sodium hydroxide in advance, polypropylenglycol diglycidyl ether was then added into the mixed solution, the mixed solution was heated to 40° C., after the mixed solution was stirred to react for 2 h, the mixed solution was kept standing for 30 min, and the mixed solution was filtered and washed; and the mixed solution was filled and sterilized to prepare 20-80 μm radionuclide ¹⁷⁷Lu microspheres (the yield of the microspheres was 55%, the radioactive activity value was 1.94 mCi, the labeling rate of nuclide ¹⁷⁷Lu was 97%, the density was 1.07 g/cm³, the BET specific surface area of the microspheres was 8.7 m2/g, and the BJH average pore diameter of the microspheres was 22.3 nm).

Infrared IR analysis (KBr pellet, cm⁻¹): 3441, 2928, 2882, 1731, 1656, 1453, 1391, 1308, 1160, 1041, 628.

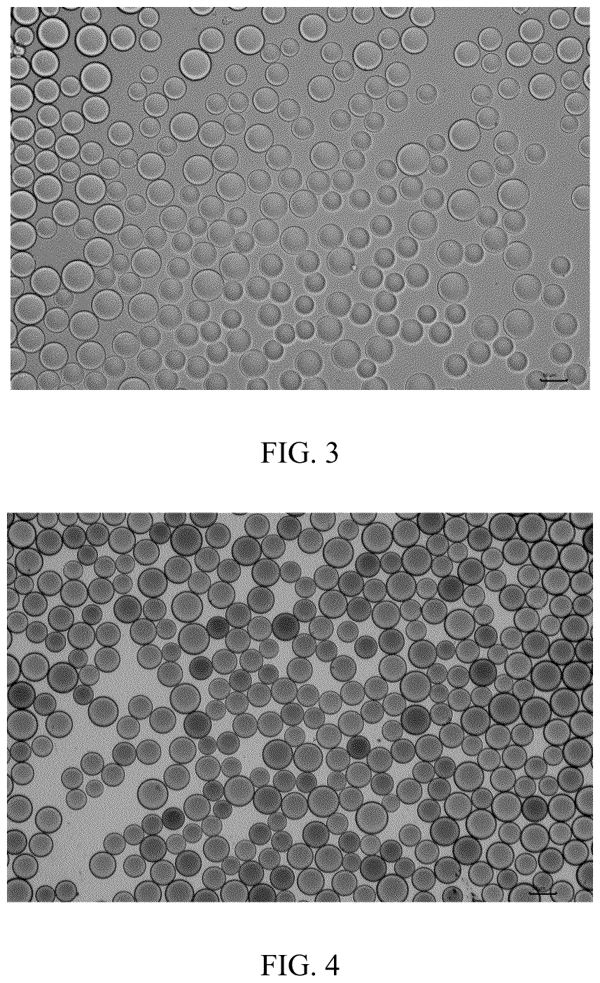

FIG. 3 is an optical microphotograph of radionuclide ¹⁷⁷Lu microspheres obtained. It could be seen from the microphotograph that the particle size of the microspheres is relatively uniform, and the microspheres are spheroidal.

### Example 4 Preparation of Y-90 Loading Nuclear Particles

(1) An emulsifier span-60 was added into petroleum ether to form an oil phase solution, where the mass percentage of the emulsifier in the oil phase solution was 0.1 wt %.

Acrylamide, 2-acrylamide-2-methyl sodium proparesulfonate, polyethylene glycol diacrylate, potassium persulfate, and tetramethyl ethylenediamine were weighed at the mass ratio of 30:60:5:2:3.

Acrylamide, 2-acrylamide-2-methyl sodium proparesulfonate, polyethylene glycol diacrylate, and potassium persulfate were mixed with water to form a mixed aqueous solution, where the mass percentage of water in the mixed aqueous solution was 65%.

The mixed aqueous solution was dropwise added into an oil phase solution in a stirring condition to obtain a mixture, the mass ratio of the mixed aqueous solution to the oil phase solution being 1:10, and the mixture was reacted at 40° C. for 2 h to form a prepolymer intermediate.
(2) A 1 mCi ⁹⁰YCl₃ solution was added into the prepolymer intermediate dispersion obtained in S(1), tetramethyl ethylenediamine was dropwise added at 50° C. to react for 3 h to obtain a product, the product was kept standing for 30 min, and the product was filtered and washed to obtain ⁹⁰Y nuclear particles. (The yield was 25%, the radioactive activity value was 0.78 mCi, and the labeling rate of nuclide ⁹⁰Y was 78%).

Infrared IR analysis (KBr pellet, cm⁻¹): 3438, 2932, 2873, 1667, 1546, 1454, 1416, 1305, 1155, 1037, 619.

### Example 5 Preparation of Y-90 Loading Nuclear Particles

(1) An emulsifier tween-80 was added into petroleum ether to form an oil phase solution, where the mass percentage of the emulsifier in the oil phase solution was 0.2 wt %.

Hydroxyethyl acrylate, sodium acrylate, N,N′-methylene diacrylamide, ammonium persulfate, and tetramethyl ethylenediamine were weighed at the mass ratio of 35:55:4:3:3.

Hydroxyethyl acrylate, sodium acrylate, N,N′-methylene diacrylamide, and ammonium persulfate were mixed with water to form a mixed aqueous solution, where the mass percentage of water in the mixed aqueous solution was 50%.

The mixed aqueous solution was dropwise added into an oil phase solution in a stirring condition to obtain a mixture, the mass ratio of the mixed aqueous solution to the oil phase solution being 1:12, and the mixture was reacted at 30° C. for 2 h to form a prepolymer intermediate.
(2) A 5 Ci ⁹⁰YCl₃ solution was added into the prepolymer intermediate dispersion obtained in S(1), tetramethyl ethylenediamine was dropwise added at 40° C. to react for 4 h to obtain a product, the product was kept standing for 30 min, and the product was filtered and washed to obtain ⁹⁰Y nuclear particles. (The yield was 35%, the radioactive activity value was 4.15 Ci, and the labeling rate of nuclide ⁹⁰Y was 83%).

Infrared IR analysis (KBr pellet, cm⁻¹): 3371, 2942, 2912, 1654, 1561, 1441, 1329, 1095, 850.

### Example 6 Preparation of Lu-177 Loading Nuclear Particles

(1) An emulsifier EM 90 was added into petroleum ether to form an oil phase solution, where the mass percentage of the emulsifier in the oil phase solution was 0.1 wt %.

Hydroxyethyl acrylate, 2-acrylamide-2-methyl sodium proparesulfonate, 3-arm-polyethylene glycol-acrylate, ammonium persulfate, and tetramethyl ethylenediamine were weighed at the mass ratio of 30:60:8:1:1.

Hydroxyethyl acrylate, 2-acrylamide-2-methyl sodium proparesulfonate, 3-arm-polyethylene glycol-acrylate, and ammonium persulfate were mixed with water to form a mixed aqueous solution, where the mass percentage of water in the mixed aqueous solution was 50%.

The mixed aqueous solution was dropwise added into an oil phase solution in a stirring condition to obtain a mixture, the mass ratio of the mixed aqueous solution to the oil phase solution being 1:8, and the mixture was reacted at 30° C. for 2 h to form a prepolymer intermediate.
(2) A 2 mCi ¹⁷⁷LuCl₃ solution was added into the prepolymer intermediate dispersion obtained in S(1), tetramethyl ethylenediamine was dropwise added at 40° C. to react for 4 h to obtain a product, the product was kept standing for 30 min, and the product was filtered and washed to obtain ¹⁷⁷Lu nuclear particles. (The yield was 32%, the radioactive activity value was 1.7 mCi, and the labeling rate of nuclide ⁹⁰Y was 85%).

Infrared IR analysis (KBr pellet, cm⁻¹): 3448, 2971, 2932, 2881, 1736, 1671, 1559, 1458, 1390, 1366, 1230, 1037, 627.

### Example 7 Preparation of Ho-166 Loading Microspheres

(1) An emulsifier OP-10 was added into petroleum ether to form an oil phase solution, where the mass percentage of the emulsifier was 0.1 wt %.

Hydroxyethyl acrylate, sodium acrylate, N,N′-methylene diacrylamide, ammonium persulfate, and tetramethyl ethylenediamine were weighed at the mass ratio of 50:40:7:2:1.

Hydroxyethyl acrylate, sodium acrylate, N,N′-methylene diacrylamide, and ammonium persulfate were mixed with water to form a mixed aqueous solution, where the mass percentage of water in the mixed aqueous solution was 50%.

The mixed aqueous solution was dropwise added into an oil phase solution in a stirring condition to obtain a mixture, the mass ratio of the mixed aqueous solution to the oil phase solution being 1:10, and the mixture was reacted at 30° C. for 2 h to form a prepolymer intermediate.
(2) A 3 mCi ¹⁶⁶HoCl₃ solution was added into the prepolymer intermediate dispersion obtained in S(1), and tetramethyl ethylenediamine was dropwise added at 40° C. to react for 4 h to obtain ¹⁶⁶Ho nuclear particles.
(3) The nuclear particles in S(2), dopamine hydrochloride, and a Tris-HCl buffer solution were weighed at the mass ratio of 1:15:25.

The nuclear particles in S(2) were added into the Tris-HCl buffer solution, then dopamine hydrochloride was added, the solution was heated to 37° C., after the solution was stirred to react for 2 h, the solution was kept standing for 30 min, and the solution was filtered and washed; and the solution was filled and sterilized to prepare 20-80 μm radionuclide ¹⁶⁶Ho microspheres (the yield of the microspheres was 47%, the radioactive activity value was 2.92 mCi, the labeling rate of nuclide ¹⁶⁶Ho was 97%, the density was 1.06 g/cm³, and the BJH average pore diameter of the microspheres was 27.6 nm).

Infrared IR analysis (KBr pellet, cm⁻¹): 3425, 2925, 1654, 1561, 1449, 1403, 1316, 1202, 1114, 849, 781, 621.

FIG. 4 is an optical microphotograph of radionuclide ¹⁶⁶Ho microspheres obtained. It could be seen from the microphotograph that the particle size of the microspheres is relatively uniform, and the microspheres are spheroidal.

### Example 8 Preparation of La-140 Loading Microspheres

(1) An emulsifier EM 90 was added into petroleum ether to form an oil phase solution, where the mass percentage of the emulsifier was 0.2 wt %.

Acrylamide, 2-acrylamide-2-methyl sodium proparesulfonate, polyethylene glycol diacrylate, ammonium persulfate, and tetramethyl ethylenediamine were weighed at the mass ratio of 40:55:2:2:1.

Acrylamide, 2-acrylamide-2-methyl sodium proparesulfonate, polyethylene glycol diacrylate, and ammonium persulfate were mixed with water to form a mixed aqueous solution, where the mass percentage of water in the mixed aqueous solution was 50%.

The mixed aqueous solution was dropwise added into an oil phase solution in a stirring condition to obtain a mixture, the mass ratio of the mixed aqueous solution to the oil phase solution being 1:10, and the mixture was reacted at 30° C. for 2 h to form a prepolymer intermediate.
(2) A 2 mCi ¹⁴⁰LaCl₃ solution was added into the prepolymer intermediate dispersion obtained in S(1), and tetramethyl ethylenediamine was dropwise added at 40° C. to react for 4 h to obtain ¹⁴⁰La nuclear particles.
(3) The nuclear particles in S(2), sodium alginate, polypropylenglycol diglycidyl ether, and a sodium hydroxide solution (concentration was 5 wt %) were weighed at the mass ratio of 1:20:2:5.

The nuclear particles in S(2) were added into a solution where sodium alginate was uniformly mixed with sodium hydroxide in advance, polypropylenglycol diglycidyl ether was then added into the mixed solution, the mixed solution was heated to 40° C., after the mixed solution was stirred to react for 2 h, the mixed solution was kept standing for 30 min, and the mixed solution was filtered and washed; and the mixed solution was cleaned, filled and sterilized to prepare 20-80 μm radionuclide ¹⁴⁰La microspheres (the yield of the microspheres was 41%, the radioactive activity value was 1.92 mCi, the labeling rate of nuclide ¹⁴⁰La was 98%, the density was 1.06 g/cm³, the BET specific surface area of the microspheres was 9.4 m²/g, and the BJH average pore diameter of the microspheres was 19.6 nm).

Infrared IR analysis (KBr pellet, cm⁻¹): 3436, 2931, 1614, 1417, 1303, 1107, 1036, 620.

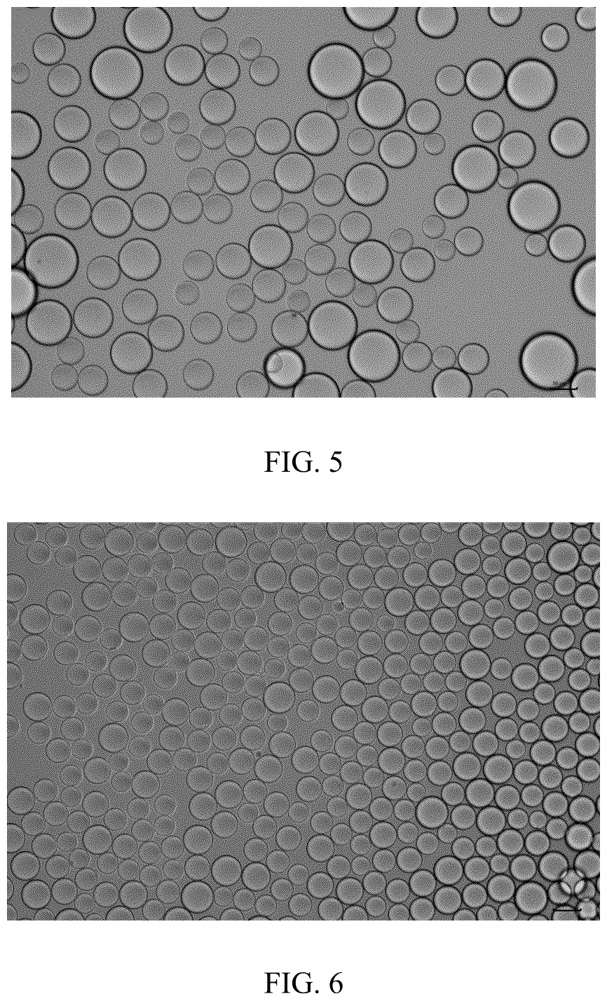

FIG. 5 is an optical microphotograph of radionuclide ¹⁴⁰La microspheres obtained. It could be seen from the microphotograph that the particle size of the microspheres is relatively uniform, and the microspheres are spheroidal.

### Example 9 Preparation of Y-90 and Lu-177 Loading Microspheres

(1) An emulsifier EM 90 was added into petroleum ether to form an oil phase solution, where the mass percentage of the emulsifier in the oil phase solution was 0.1 wt %.

Hydroxyethyl acrylate, 2-acrylamide-2-methyl sodium proparesulfonate, 3-arm-polyethylene glycol-acrylate, ammonium persulfate, and tetramethyl ethylenediamine were weighed at the mass ratio of 30:60:8:1:1.

Hydroxyethyl acrylate, 2-acrylamide-2-methyl sodium proparesulfonate, 3-arm-polyethylene glycol-acrylate, and ammonium persulfate were mixed with water to form a mixed aqueous solution, where the mass percentage of water in the mixed aqueous solution was 50%.

The mixed aqueous solution was dropwise added into an oil phase solution in a stirring condition to obtain a mixture, the mass ratio of the mixed aqueous solution to the oil phase solution being 1:8, and the mixture was reacted at 30° C. for 2 h to form a prepolymer intermediate.
(2) A 3 Ci ⁹⁰YCl₃ solution and a 2 Ci ¹⁷⁷LuCl₃ solution were added into the prepolymer intermediate dispersion obtained in S(1) in sequence, and tetramethyl ethylenediamine was dropwise added at 40° C. to react for 4 h to obtain ⁹⁰Y and ¹⁷⁷Lu nuclear particles.
(3) The nuclear particles in S(2), sodium alginate, polyethylene glycol bisglycidyl ether, and a sodium hydroxide solution (concentration was 5 wt %) were weighed at the mass ratio of 1:20:2:5.

The nuclear particles in S(2) were added into a solution where sodium alginate was uniformly mixed with sodium hydroxide in advance, polyethylene glycol diglycidyl ether was then added into the mixed solution, the mixed solution was heated to 40° C., after the mixed solution was stirred to react for 2 h, the mixed solution was kept standing for 30 min, and the mixed solution was filtered and washed; and the mixed solution was cleaned, filled, and sterilized to prepare 20-80 μm radionuclide ⁹⁰Y microspheres and ¹⁷⁷Lu microspheres (the yield of the microspheres was 47%, the radioactive activity value of ⁹⁰Y was 2.94 Ci, the radioactive activity value of ¹⁷⁷Lu was 1.95 Ci, the labeling rate of nuclide ⁹⁰Y was 98%, the labeling rate of nuclide ¹⁷⁷Lu was 97%, the density was 1.06 g/cm³, the BET specific surface area of the microspheres was 8.6 m²/g, and the BJH average pore diameter of the microspheres was 21.4 nm).

Infrared IR analysis (KBr pellet, cm⁻¹): 3444, 2931, 1730, 1653, 1569, 1407, 1301, 1180, 1038, 627.

FIG. 6 is optical microphotographs of radionuclide ⁹⁰Y and ¹⁷⁷Lu microspheres obtained. It could be seen from the microphotograph that the particle size of the microspheres is relatively uniform, and the microspheres are spheroidal.

### Example 10 Radionuclide ⁹⁰Y Microspheres Loading Doxorubicin Hydrochloride

1 g of the radionuclide ⁹⁰Y microspheres (the radioactive activity value was 150 mCi) prepared in example 2 was taken and added into 50 mg of a doxorubicin hydrochloride solution (the concentration was 20 mg/mL), the solution was mixed and kept standing, a supernatant solution was taken at 15 min, the concentration of doxorubicin hydrochloride in the solution was measured with HPLC, and a drug loading capacity was calculated according to a difference between concentrations of doxorubicin hydrochloride before and after loading the drug. The drug loading ratio of ⁹⁰Y microspheres on doxorubicin hydrochloride at 15 min may reach 95%, and the drug loading capacity is 47.5 mg/g microspheres.

### Example 11 Radionuclide ⁹⁰Y Microspheres Loading Bevacizumab

1 g of the radionuclide ⁹⁰Y microspheres (the radioactive activity value was 50 mCi) prepared in example 2 was taken and added into 60 mg of a bevacizumab solution (the concentration was 3 mg/mL), the solution was mixed and then kept standing, a supernatant solution was taken at 15 min, the concentration of bevacizumab in the solution was measured with ELIASA, and a drug loading capacity was calculated according to a difference between concentrations of bevacizumab before and after loading the drug. The drug loading ratio of ⁹⁰Y microspheres on bevacizumab at 15 min may reach 75%, and the drug loading capacity is 45 mg/g microspheres.

### Example 12 Nuclide Drop Rate Test

A nuclide drop rate test was performed with the radionuclide microspheres prepared in examples 1-9:1.00 g of the radionuclide microspheres (wet microspheres) was weighed and placed in a penicillin bottle, 5 ml of a 0.9% normal saline solution was added into the penicillin bottle, the penicillin bottle was sealed and placed in a constant temperature oven, and a constant temperature program was set to operate; a sample was taken out at a test time point, the temperature of the sample was decreased to room temperature, then the sample was centrifugalized, a supernate with a certain volume was taken for a radioactivity test, and the drop rate of nuclides of the microspheres was calculated. The nuclide drop rates of the microspheres on the 0^{th} day, the 15^{th} day, and the 30^{th} day were studied at 58° C. Results are shown in Table 1.

TABLE 1

| | Experimental condition | | |
|---|---|---|---|
| | The 0^{th} day | The 15^{th} day | The 30^{th} day |
| Number | (58° C.) | (58° C.) | (58° C.) |
| Example 1 | 0.018% | 0.019% | 0.022% |
| Example 2 | 0.006% | 0.007% | 0.007% |
| Example 3 | 0.023% | 0.024% | 0.028% |
| Example 4 | 0.047% | 0.071% | 0.117% |
| Example 5 | 0.037% | 0.048% | 0.093% |
| Example 6 | 0.042% | 0.085% | 0.162% |
| Example 7 | 0.031% | 0.033% | 0.036% |
| Example 8 | 0.043% | 0.044% | 0.046% |
| Example 9 | 0.032% | 0.035% | 0.039% |

It could be seen from the results of Table 1 that the nuclide drop rates of the microspheres prepared in example 4, example 5 and example 6 without the secondary crosslinking polymerization step are obviously higher than those in other examples, indicating that secondary crosslinking polymerization of the small molecule monomers or the high molecular materials at the centers of the nuclear particles may effectively reduce the nuclide drop rate in the microspheres.

To sum up, the radionuclides are polymerized in a coordinated manner in the microspheres by using the functional monomer containing carboxylic acid groups or sulfonic acid groups to prepare the radionuclide microspheres. The radioactive microspheres provided by the present disclosure may load various nuclides at the same time and chemotherapeutic drugs and polypeptide biological drugs as well. The radionuclide microspheres featuring a small microsphere density, high nuclide loading efficiency, a large loading capacity, a low drop rate, high safety and a simple preparation process may be used for treating solid malignant tumors such as liver cancer and lung cancer.

Compared with the related art, taking the hydrogel microspheres containing active adsorption groups as a carrier, the radionuclides are coordinated and packaged in the hydrogel microspheres to prepare the radionuclide microspheres. The radionuclide microspheres may load various nuclides at the same time and chemotherapeutic drugs and polypeptide biological drugs as well. The density of the microspheres is close to that of blood. The radionuclide microspheres feature a good suspension property, are easy to deliver, and are easier to arrive at vascular endings during vascular intervention. The radionuclide microspheres feature high radionuclide load efficiency, a large loading capacity, a low drop rate, high safety, and a simple preparation process. The microspheres may be implanted via vascular intervention and percutaneous puncture and may be used for treating solid malignant tumors such as liver cancer and lung cancer.

The radionuclide microspheres, the preparation method therefor and the application thereof provided by the present disclosure are described in detail above.

Any apparent alteration made on the present disclosure by persons of ordinary skilled in the art without departing from the substantive content of the present disclosure shall constitute an infringement of the patent right of the present disclosure, and the person shall bear corresponding legal liability.

What is claimed is:

1. Radionuclide microspheres, comprising: at least one or more radionuclides, being microspheres loading the radionuclides,
wherein the radionuclide microspheres are formed by polymerizing nuclear particles and one or more small molecule monomers or high molecular materials, the nuclear particles being formed by coordination polymerization of radionuclides and a prepolymer intermediate; and
the prepolymer intermediate is formed by polymerizing a structural monomer, a functional monomer and a vinyl crosslinker, wherein the mass ratio of the structural monomer, the functional monomer, and the vinyl crosslinker is 1:(0.01-8):(0.01-2); and the particle size of the microspheres is 5-200 μm.

2. The radionuclide microspheres according to claim 1, wherein
the structural monomer is a compound containing hydrophilic functional groups such as hydroxyl, amino and carboxyl;
the functional monomer is an organic salt with carboxylic acid groups or sulfonic acid groups;
the vinyl crosslinker is a water soluble acrylamide or acrylate compound; and
the small molecule monomer or high molecular material is a water soluble compound.

3. The radionuclide microspheres according to claim 2, wherein
the structural monomer is selected from one or more of acrylamide, methacrylamide, acrylic acid, methacrylic acid, hydroxyethyl acrylate, hydroxyl propyl acrylate, halogenated acrylic acid, and halogenated methacrylic acid;
the functional monomer is selected from one or more of sodium acrylate, acryloylamino sodium ethyl carboxylate, 2-acrylamide-2-methyl sodium propyl carboxylate, and 2-acrylamide-2-methyl sodium propanesulfonate;
the vinyl crosslinker is selected from one or more of N,N′-methylene diacrylamide, N,N′-ethylenebisacrylamide, polyethylene glycol diacrylate, polypropylene glycol acrylate, 3-arm-polyethylene glycol-acrylamide, 3-arm-polyethylene glycol-acrylate, 4-arm-polyethylene glycol-acrylamide, and 4-arm-polyethylene glycol-acrylate;
the small molecule monomer is selected from one or more of acrylamide, phosphonitrile, and dopamine; and
the high molecular material is formed by a reaction on one or more of gelatin, sodium alginate, sodium hyaluronate, carboxymethyl chitosan, polyvinyl alcohol, polythreonine and polyserine, and butanediol diglycidyl ether, polyethylene glycol diglycidyl ether or polypropylenglycol diglycidyl ether.

4. The radionuclide microspheres according to claim 1, wherein
the radionuclide is selected from at least one of lanthanum, yttrium, technetium, strontium, praseodymium, samarium, europium, gadolinium, terbium, zirconium, holmium, erbium, ytterbium, lutetium, rhenium, and gallium.

5. A preparation method of the radionuclide microspheres according to claim 1, comprising the following steps:
S1: performing a reaction on the structural monomer, the functional monomer, and the vinyl crosslinker to prepare the prepolymer intermediate;
S2: performing coordination polymerization on metal ions of the radionuclides in the prepolymer intermediate through a coordination interaction between the prepolymer intermediate and the one or more radionuclides to prepare nuclear particles; and
S3: performing a secondary polymerization reaction on the nuclear particles and the one or more small molecule monomers or high molecular materials to obtain a product, and cleaning, filling, and sterilizing the product to prepare the radionuclide microspheres.

6. The preparation method according to claim 5, wherein steps S1, S2, and S3 are specifically operated as follows:
S61: placing an emulsifier in an oil phase solvent to form an oil phase solution, the oil phase solvent comprising: petroleum ether, n-heptane, cyclohexane and/or liquid paraffin;
S62: mixing the structural monomer, the functional monomer, the vinyl crosslinker and an initiator with water to form a mixed aqueous solution;
S63: dropwise adding the mixed aqueous solution prepared in S62 into the oil phase solution prepared in S61 in a stirring condition to react to prepare the prepolymer intermediate;
S64: dropwise adding the solution containing the radionuclide ions into the prepolymer intermediate prepared in S63 in a stirring condition, and dropwise adding a catalyst tetramethyl ethylenediamine to react to prepare the nuclear particles;
S65: dropwise adding the small molecule monomers or high molecular materials into the nuclear particle solution prepared in S64 in a stirring condition for a further secondary polymerization reaction to obtain a product, and cleaning, filling, and sterilizing the product to prepare the radionuclide microspheres.

7. The preparation method according to claim 6, wherein
the emulsifier is selected from one or more of OP-10, EM90, span-60, and tween-80, and the mass ratio of the emulsifier to the oil phase solvent is 1:(20-1000);
the initiator comprises one or more of potassium persulfate, sodium persulfate, and ammonium persulfate;
the mass ratio of the structural monomer, the functional monomer, the vinyl crosslinker, and the initiator is 1:(0.01-8):(0.01-2):(0.01-1);
in S62, the mass ratio of the total mass of the structural monomer, the functional monomer, the vinyl crosslinker, and the initiator to water is 1:(0.3-3);
in S63, the mass ratio of the mixed solution prepared in S62 to the oil phase solution prepared in S61 is 1:(8-15); and
in S65, the mass ratio of the nuclear particle solution prepared in S64 to the small molecule monomers is 1:(0.1-20); the high molecular material is formed by the reaction on one or more of gelatin, sodium alginate, sodium hyaluronate, carboxymethyl chitosan, polyvinyl alcohol, polythreonine and polyserine, and butanediol diglycidyl ether, polyethylene glycol diglycidyl ether or polypropylenglycol diglycidyl ether; and the mass ratio of the nuclear particle solution prepared in S64 to the high molecular materials is 1:(1-20).

8. The preparation method according to claim 6, further comprising:
S66: adsorbing, by the radionuclide microspheres prepared in S65, positively charged drugs through an electrostatic interaction, or physically adsorbing large molecular drugs, preferably bevacizumab, PD-1 or PD-L1, via hydrogen bonds.

9. An application of radionuclide microspheres in preparing drugs for treating solid malignant tumors, wherein
the radionuclide microspheres are the radionuclide microspheres according to claim 1.
